(19) United States
(12) Patent Application Publication
(10) Pub. No.: US 2026/0305949 A1
(43) Pub. Date: Oct. 8, 2026

(54) SYSTEM, METHOD AND COMPUTER-ACCESSIBLE MEDIUM FOR PROVIDING OPTICAL PLAQUE BURDEN ASSESSMENT

(71) Applicant: KONINKLIJKE PHILIPS N.V., Eindhoven (NL)

(72) Inventors: Dave Vader, Concord, MA (US); James Mclean, Somerville, MA (US); Jason Tucker-Schwartz, Melrose, MA (US); Eman Namati, Concord, MA (US); Damon Depaoli, Ottawa (CA); Ying Mu, Bedford, MA (US); Daniel Gourdeau, Medford, MA (US); Vivek Walimbe, Westford, MA (US)

(21) Appl. No.: 19/483,497

(22) PCT Filed: May 16, 2024
(86) PCT No.: PCT/US2024/029614

§ 371 Date: Nov. 11, 2025

Related U.S. Application Data
(60) Provisional application No. 63/466,773, filed on May 16, 2023.

(51) Int. Cl. A61B 5/02; A61B 1/00; A61B 1/313; A61B 5/00; A61B 5/107; G06T 7/00; G06T 7/62; G06T 7/70

(52) U.S. Cl. CPC A61B 5/02007; A61B 1/0005; A61B 1/3137; A61B 5/0066; A61B 5/1076; G06T 7/0012; G06T 7/62; G06T 7/70; G06T 2200/24; G06T 2207/10101; G06T 2207/20081; G06T 2207/30048; G06T 2207/30101

(57) ABSTRACT

Exemplary embodiments of method, system and computer-accessible medium can be provided for measuring certain exemplary clinical features that can direct clinical deceeeision making during percutaneous coronary intervention (PCI), such as, e.g., plaque burden—critical for assessing the health of a vessel and the vulnerability of a patient or plaque—and an external elastic lamina (EEL) diameter—critical for stent, balloon, and other coronary vessel modification tool sizing and positioning during PCI. Thus, it is possible to provide exemplary method, system and a computer-accessible medium using which it is possible to obtain an image of at least one section of the arterial wall, automatically detect one or more visible portions of an arterial external elastic laminae of the section(s), automatically determine or estimate one or more invisible portions of the arterial external elastic laminae based on the automatically detected visible portion(s), and automatically combine the visible and invisible portions to provide the measurement(s) of the external elastic laminae.

### CROSS REFERENCE TO RELATED APPLICATION(S)

This application relates to and claims the benefit of priority from U.S. Provisional Patent Application No. 63/466,773, filed on May 16, 2023, the entire disclosure of which is incorporated herein by reference in its entirety.

### FIELD OF THE DISCLOSURE

The present disclosure relates generally to analysis of plaque, and more particularly, to systems, methods, and computer-accessible medium for providing a plaque burden assessment.

### BACKGROUND INFORMATION

Coronary arteries are a layered structure. Layers such as external elastic lamina (EEL), or external elastic media (EEM), in most clinical cases can represent the true size of coronary vessel for treatment planning and is therefore an important tissue structure to assess during the planning of a percutaneous coronary intervention (PCI) procedure. Stent, balloon, and other coronary vessel modifications methods can benefit from planning, sizing and positioning using a measured EEL diameter, compared to sizing using a stenotic lumen which no longer represents the true vessel size in an atherosclerotic lesion. The EEL to lumen ratio can also be an important metric for assessing vessel health and vulnerability to future adverse events. The EEL, however, in the presence of atherosclerosis, is a sub-surface tissue structure and has been traditionally difficult to visualize & measure using optical methods, making intravascular ultrasound that suffers from reduced image resolution a preferred methodology for vessel sizing.

Intravascular ultrasound (IVUS) is effective for imaging deep tissue structures, due to the nature of the ultrasound imaging modality. However, IVUS provides poor spatial resolution compared to some optical modalities and therefore provides incomplete data when assessing & sizing arterial structures important for clinical decision making such as thrombus and dissections. Providing the resolution of optical sensing modalities (e.g., intravascular optical coherence tomography (IVOCT)) as well as the ability to measure relatively deep structures such as the EEL would be of significant value in making important clinical decisions during PCI.

Accordingly, there is a need to provide apparatuses, systems, computer-accessible medium and/or methods to address and/or overcome at least some of such deficiencies. Furthermore, providing technology to infer layered structure positions, for instance when it is not directly visible in an image can provide a beneficial clinical utility.

### SUMMARY OF EXEMPLARY EMBODIMENTS

According to exemplary embodiments of the present disclosure, it is possible to overcome the challenges of using optical imaging to detect and assess the position and size of the layered structures (e.g., intima, media, EEL). To that end, it is possible to provide exemplary embodiments of methods, systems and computer-accessible medium for measuring certain exemplary clinical features that can direct clinical decision making during percutaneous coronary intervention (PCI), such as, e.g., plaque burden—critical for assessing the health of a vessel and the vulnerability of a patient or plaque—and EEL diameter—critical for stent, balloon, and other coronary vessel modification tool sizing and positioning during PCI.

The methods, systems and computer-accessible medium according to the exemplary embodiments can be used to visualize and estimate the position and size of the EEL, e.g., in both two and three dimensions, along with downstream clinical features, using intravascular imaging (e.g., ultrasound imaging, e.g., optical imaging). In one exemplary embodiment, an enhanced visualization of the EEL is achieved via a high-sensitivity optical coherence tomography (OCT) imaging system with improved depth imaging. In some exemplary embodiments, image processing procedures to automatically detect visible portions (e.g., segment, e.g., using machine-learning/deep-learning procedures), optionally pre-process (e.g., center, register, enhance, de-noise, etc.), and infer (e.g., interpolate, extrapolate, estimate, etc.) visibly obscured or invisible portions, of EEL or any other layered coronary structure. In some exemplary embodiments of the present disclosure, enhanced depth imaging can be combined with exemplary image processing methods to provide a further EEL localization accuracy. In some exemplary embodiments of the present disclosure, such improved visualization, detection, pre-processing and inference can result in highly accurate EEL diameter measurements which are critical for guiding PCI. In some exemplary embodiments according to the present disclosure, such improved visualization, detection, pre-processing and inference can result in highly accurate plaque burden estimates, which are critical for an adverse event risk assessment (e.g., at the patient or plaque level).

Thus, exemplary method, system and a computer-accessible medium according to exemplary embodiments of the present disclosure can be provided using which it is possible to obtain an image of at least one section of the arterial wall, automatically detect one or more visible portions of an arterial external elastic laminae of the section(s), automatically determine or estimate one or more invisible portions of the arterial external elastic laminae based on the automatically detected visible portion(s), and automatically combine the visible and invisible portions to provide the measurement(s) of the external elastic laminae.

For example, it is possible to automatically estimate a plaque burden from at least one of the one or more first portions or the one or more second portions. With, e.g., a display device, it is possible to display a representation of the external elastic laminae or the plaque burden based on a viewport within a user interface, e.g., upon reaching a predetermined threshold of the plaque burden. The intravascular image(s) can be optical coherence tomography (OCT) image(s) received from an OCT system with a sensitivity that is greater than about 100 dB. the one or more further (e.g., invisible) portions can be determined or inferred using a machine learning procedure. The machine learning procedure can determine or infer the second (e.g., invisible) portions by an interpolation from neighboring frames.

According to another exemplary embodiment of the present disclosure, methods, systems and computer-accessible medium can be provided for determining or providing one or more measurements of an arterial wall. Thus, it is possible to, e.g., obtain an image of at least one section of the arterial wall, automatically detect one or more first portions of an arterial external elastic laminae of the section(s) of the arterial wall, automatically detect one or more second portions of the arterial wall; and determine or estimate a plaque burden by processing the detected first portion(s) and the second portions(s). For example, it is also possible to display a representation of the plaque burden on a viewport of a user interface, e.g., when a predetermined threshold of the plaque burden is reached.

According to still another exemplary embodiment of the present disclosure, methods, systems and computer-accessible medium can be provided for determining or providing one or more measurements of a coronary artery. Thus, it is possible to, e.g., obtain an image of at least one portion of the coronary artery, and, with a machine learned procedure, directly or estimate at least one measure of a plaque burden based on one or more features or information for the portion(s) of the image.

According to a further exemplary embodiment of the present disclosure, methods, systems and computer-accessible medium can be provided for determining or providing one or more measurements of a coronary artery. Thus, it is possible to, e.g., obtain an image of at least one portion of the coronary artery, and, with a machine learned procedure, determine or estimate ae position the external elastic laminae within the at least one portion of the image. For example, the image can be generated using a first imaging modality. It is possible to train the machine learned procedure to determine or estimate the position of the external elastic laminae using registered data from a second image modality which is different from the first image modality.

In one exemplary embodiment, the registered data can have a resolution lower (e.g., IVUS) or higher (e.g., histology, e.g., confocal imaging) than that of the image from the first imaging modality (e.g., IVOCT). The machine learned procedure can determine or estimate the position of the external elastic laminae in areas where the external elastic laminae is invisible within the image. The machine learned procedure can generate further images which are adjacent to the image.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an illustration of a cross section of a coronary vessel to explain plaque burden;

FIG. 2 is a block diagram of a workflow and a method according to an exemplary embodiment of the present disclosure;

FIG. 3 is a set of exemplary illustration of atherosclerotic plaque and risk associated with plaque burden versus a higher plaque burden, higher risk of atherosclerotic/vulnerable plaque;

FIG. 4 is an exemplary illustration of an exemplary interpolation of EEL contour using sections of automatically detected/visible EEL;

FIG. 5 is an exemplary illustration of centering of EEL prior to interpolation;

FIG. 6 is an exemplary illustration of ND Interpolation of EEL;

FIG. 7 is an exemplary illustration of ND Interpolation of EEL using an ellipticity constraint, optimized/trained using IVUS or ground truth data;

FIG. 8 is an exemplary illustration of ND interpolation within continuous regions, defined by automatically detected side-branches;

FIG. 9 is an exemplary illustration of an exemplary UI. Multi-panel. EEL overlays along with thresholded plaque burden measurement overlays greater than a pre-defined value;

FIG. 10 is a diagram of an exemplary Intravascular Imaging System according to an exemplary embodiment of the present disclosure; and

FIG. 11 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended paragraphs.

### DETAILED DESCRIPTION OF EXEMPLARY EMBODIMENTS

In some exemplary embodiments according to the present disclosure, position and size of the EEL is inferred directly via a frame-based (e.g., image-based) or volume-based (e.g., multi-frame, multi-image, etc.) machine learning procedure, which can take as input a frame or volume of data or a pre-processed version of said data (e.g., centered, registered, enhanced, de-noised, etc.), in addition to physical parameters associated with image acquisition and/or clinical parameters associated with a patient. In some exemplary embodiments of the present disclosure, position and size of the EEL can be inferred indirectly via a frame-based or volume-based machine learning procedure, which initially outputs the areas where EEL is clearly visible. A custom shape-fitting model can then be used in each frame to interpolate and/or extrapolate the position and size of the EEL when it is not directly visible circumferentially.

According to some exemplary embodiments of the present disclosure, the shape-fitting model can utilize visible EEL data from multiple frames to interpolate and/or extrapolate the position and size of the EEL in a single target frame. In some exemplary embodiments of the present disclosure, priori information such as a normative atlas, or a patient specific atlas, (e.g., generated by another imaging modality, such as, for example, by coronary computed tomography angiography-CCTA) for a given vessel can be used to inform a pre-processing and/or an interpolation and/or inference procedure. In some exemplary embodiments according to the present disclosure, additional physiological information on the vessel (e.g., ischemia index, e.g., local FFR measurements, e.g., longitudinal FFR measurements, e.g. location of side branches, vessel of interest such as RCA, LCX, etc.) can be used to further specialize the machine learning models and/or any of the associated processing methods (shape-fitting, etc.) can be utilized with the exemplary embodiments of the present disclosure.

According to various exemplary embodiment of the present disclosure, an exemplary EEL detection can be inferred/interpolated using a shape-fitting model (e.g., a circular or elliptical fit) with a defined constraint (e.g., an ellipticity constraint). In some exemplary embodiments according to the present disclosure, an e.g., elliptical fit model may use an optimized fit constraint (e.g., a maximum ellipticity, e.g., a maximum diameter) and said optimized constraint may update automatically based on some automatically detected feature (e.g., EEL diameter). Furthermore, plaques of varying composition have varying levels of hardness. For example, calcium plaques are hard & crystalized structures, while lipid pools can be soft. In some exemplary embodiments, an optimized fit constraint (e.g., ellipticity constraint, e.g., an eccentricity constraint) is automatically updated for an automatically detected plaque type (e.g., calcium or lipid-rich). Moreover, lumen narrowing of a coronary vessel as well as other morphological characteristics (e.g., tortuosity) can provide information on the potential deformation of the EEL. For example, a highly eccentric plaque that causes an eccentric lumen narrowing can cause the EEL to expand non-concentrically on the side of the plaque (possibly being narrowing). In some exemplary embodiments of the present disclosure, lumen morphology and/or other vessel morphology characteristics can be used to inform an optimized fit constraint (e.g., ellipticity constraint, an eccentricity constraint, ellipse center, etc.) and/or any other processing technique that does not include require constraint (e.g., detection, pre-processing, inference, etc.).

Patient and artery categories can influence the diameter of a given artery and the rate of the decrease of diameter along the length of a vessel. In some exemplary embodiments, an optimized fit constraint is automatically updated for a known or detected category of patient (e.g., history of CAD) or artery (e.g., left anterior descending (LAD), right coronary artery (RCA), etc.).

Aspects of the arterial lumen, e.g., the eccentricity of an automatically detected lumen, can also assist with an inference of the diameter and eccentricity of an automatically detected EEL. In some exemplary embodiments of the present disclosure, an optimized fit constraint can be automatically updated based on lumen profiles (e.g., lumen diameters or for example, lumen eccentricity, lumen indentation, lumen center, etc.).

Interpolating an automatically detected portion of an EEL can be achieved using a machine-learned procedure or a machine-learning procedure (e.g., a machine-learned elliptical fit procedure, machine-learned semantic segmentation, machine learned detection, etc.). In some exemplary embodiments according to the present disclosure, an optimized fit constraint can be a machine-learned constraint (e.g., via training on a registered IVUS training dataset, on a histology training dataset, etc.). In some exemplary embodiments of the present disclosure, the machine learning procedure can be trained on expert annotations. In some exemplary embodiments of the present disclosure, the expert annotations can include annotations only where EEL is directly visible within the intravascular imaging modality. In some exemplary embodiments of the present disclosure, the expert annotations can include annotations, whereas EEL is not directly visible and can be human-inferred (e.g., human-interpolated) based on the annotators expert technical knowledge of EEL morphology. In some exemplary embodiments of the present disclosure, the machine learning procedure can be trained on registered images from a different imaging modality from the intravascular imaging modality (e.g., registered higher resolution images, registered lower resolution images, registered histology images, registered IVUS images, registered OCT images, registered CCTA images, etc.).

Information (e.g., automatically detected objects) from neighboring frames can also be utilized to assist with the inference of a fit constraint for a specific frame. For example, in some exemplary embodiments of the present disclosure, an inference method or procedure and/or an optimized fit constraint can be automatically updated based on, at least partially, the proximity of automatically detected vascular features (e.g., side branches) within a given frame or in neighboring frames.

For example, any optimization over a fit constraint can be performed to optimize the accuracy of a plaque burden measurement.

According to further additional or alternative exemplary embodiments of the present disclosure, the user interface can summarize to the user a set of EEL measurements, potentially alongside other metrics (e.g. lumen size, lipid, etc.) to inform clinical decisions. Exemplary measurements and indicators can include any of EEL diameter, area, plaque burden, etc. and when any of the measurements fall above or beneath a computer or user-generated threshold value. For example, most or all plaque burden over about e.g., 50% can trigger a visual indicator to the user, and/or a plaque burden less than about e.g., 50% can be indicated as a recommended location to end a stent or balloon (e.g., indicated on an angiography viewport, indicated on a longitudinal viewport, indicated in a transverse intravascular viewport, etc). According to further additional or alternative exemplary embodiments of the present disclosure, the user interface can display a 3D rendering of any of such measurements.

In additional or alternative exemplary embodiments of the present disclosure, the exemplary system, method and computer-accessible medium can be used to summarize completeness of treatment using the information characterized herein, for example a residual plaque burden outside of a stent less than about e.g., 50%. For example, the EEL measurements, including, e.g., vessel size and plaque burden, can be used to directly guide PCI planning as recommended by the system, including location to start and end a stent, diameter and length of the stent, dilation balloon diameter and length.

According to yet further or alternative exemplary embodiments of the present disclosure, the exemplary system, method and computer-accessible medium can include and/or utilize procedures and/or user interface on board a mobile or integrated piece of hardware designed to acquire, process, and provide or otherwise display findings from an intravascular imaging system (e.g., an optical coherence tomography intracoronary imaging system) to a physician mid-procedure during PCI. For example, EEL measurements can be co-located to a number of other intravascular sensed parameters including lumen size, calcium size and location, lipid size and location, stent position, side-branches, and summarized to a clinician user to guide PCI decision making and confirm completeness of treatment. In addition, or alternatively, EEL measurements including, e.g., plaque burden, detected alongside other intravascular sensed parameters (e.g. cap thickness, lipid) can be used to identify the need to treat an area within a vessel. The exemplary system, method and computer-accessible medium—when using these exemplary measurements—can recommend incremental treatment with stent, scaffold, or some other methodology to proactively treat a plaque at high risk of future adverse event. Recommendations can be based on a machine learning procedure or a score-based threshold system. In some exemplary embodiments of the present disclosure, any recommendation provided by the exemplary system and/or method can be interpreted and conveyed to a user through an off-the-shelf or custom-trained large language model (LLM) which, for example, also access to intermediate features (e.g., hidden features) of any other exemplary procedure pipeline also deployed on the exemplary system and/or method.

In additional or alternative exemplary embodiments of the present disclosure, a user determined clinical guidance threshold can be used to filter EEL measurements, including plaque burden. When EEL measurements exceed or fall below the threshold, the user can be notified by the user interface via e.g., a color bar, shaded regions, text, or other visual indicator. For example, EEL measurements can be presented to the user in a viewport (e.g., multiple viewports) to guide clinical decision making. Example viewports can include a cross-sectional, longitudinal, or other plane of the intravascular modality. Exemplary measurements can also be visually overlayed to other views of co-registered modalities including angiography, summary measurement viewports (e.g., risk viewports), computed tomography, etc. Further or alternatively, e.g., EEL measurements can be co-located to other intravascular sensed parameters in a display that summarizes key insights to the physician. Combined insights can include the co-localization of large or small vessel size, high or low plaque burden, large or small lumen size, presence or absence and amount of lipid, thin or thick fibrous cap, presence or absence and amount of calcium, and other clinically meaningful combinations.

In some exemplary embodiments of the present disclosure, a pre-processing step/procedure aimed at spatially registering neighboring frames can be first used (e.g., using the center position of a partially detected EEL), prior to using multiple neighboring frames to interpolate/extrapolate the position and size of the EEL in a target frame. In some exemplary embodiments according to the present disclosure, an interpolation or extrapolation step includes 3D topological interpolation. In some exemplary embodiments of the present disclosure, an interpolation or extrapolation step includes multiple frames of intravascular imaging data. In some exemplary embodiments of the present disclosure, an interpolation or extrapolation step can include multiple frames of intravascular imaging data, whereas each frame can include only partial sections of a detected EEL.

According to further exemplary embodiments of the present disclosure, a prior knowledge of the physical and optical properties of tissue, blood and flush medium (e.g. optical attenuation, index of refraction, etc.) can be used to pre-process the input data or post-process the intermediate or final results.

In some exemplary embodiments of the present disclosure, multi-modality data (e.g. OCT and IVUS, OCT, NIRS, IVUS and NIRS, e and/or angiography and/or FFR) can be used to pre-process the input data or post-process the intermediate or final results of a plaque burden estimation procedure, and/or in some cases can be used directly for detection/inference of EEL locations(e.g., 2D detection/inference, e.g., 3D detection/inference). In some exemplary embodiments of the present disclosure, a composition of plaque can be determined from a one imaging modality (e.g., angiography) and this information can be used to refine EEL estimations within the intravascular imaging modality (e.g., OCT). In some exemplary embodiments of the present disclosure, composition of plaque (e.g., lipid concentration, lipid sub-typing, etc.) can be determined from a spectroscopic modality, e.g., NIRS, image, and this information can be used to refine estimations. In another exemplary embodiment of the present disclosure, a prior knowledge or an inferred knowledge of tissue (e.g., automatically detected tissue) properties (e.g. mechanical properties, elasticity, shear modulus, stress, strain) can be used to pre-process input data for an estimation procedure and/or post process the intermediate or final results.

Transfer learning procedure(s) can be used to take EEL measurements from imaging modalities with complimentary EEL visibility and infer the location of EEL in a different imaging modality. This can be performed by training a neural network to learn the relationship between EEL shape and structure with two different imaging modalities, where one modality may have less visibility than the other. Trans-UNet architectures with the ability to capture high-level semantic context are well suited for this task. Partially visible EEL can be inferred in 360 degrees using only the visible portions of EEL in an image. This can be done using a highly contextualized neural network trained on learning the shape of the EEL in the presence of other AI detected structural features such as lumen and calcium. This technique can be further enhanced by combining with image information using a multi-channel input.

According to additional exemplary embodiments of the present disclosure, exemplary procedures designed to detect other plaque features (e.g. calcium, lipid, TCFA, cholesterol) can be used to further refine the local properties of the plaque and guide the inference of the position and size of the EEL. In some exemplary embodiments according to the present disclosure, templates of similar transverse sections from a-priori data can be used to assist in the refinement (e.g., template matching to a similar transverse section from, for example, histology). In some exemplary embodiments of the present disclosure, plaques are automatically detected and typed based on imaging data (e.g., NIRS data, OCT Data, Angiography data, etc.).

In some exemplary embodiments of the present disclosure, high sensitivity OCT (e.g., >100 dB) can be deployed to image deeper into and arterial wall, and the extended depth of imaging allows for enhanced visualization of EEL. For example, image processing procedures (e.g., which can that utilizes a generative adversarial network (GAN)) can be applied to an OCT image to improve EEL visibility and automated EEL detection (e.g., improving plaque burden measurements). In another example, image processing procedure(s) can include image de-noising (e.g., using a custom machine learned denoising procedure, a machine learned diffusion model, etc.) to enhance the visualization of an image and/or specific features. In another example, in some exemplary variants, a pre-processed image for improved image depth visualization is provided into a machine learning procedure (e.g., a deep learning procedure, e.g., a U-Net, e.g., a Res-Net, a transformer network, a diffusion model) to improve EEL detection (e.g., EEL segmentation). In some exemplary embodiments of the present disclosure, a machine learned or machine learning procedure (e.g., a regression model) detects (e.g., measures) plaque burden (e.g., plaque burden percentage, plaque burden index,, 0-100%, 0-1, etc.) directly from an image (e.g., an IVUS image, an OCT image, etc.), or a set of images (e.g., a set of angiography images, a set of CCTA, etc.).

According to further exemplary embodiments of the present disclosure, the automated detection procedures (e.g., automated segmentation of EEL, e.g., automated detection and typing of plaques), the pre-processing procedures (e.g., image-to-image registration, image denoising, image enhancing, edge filtering, etc.), 3D interpolation methods (e.g., automatic pre-centering of frames based on EEL portions,, automatic pre-centering of frames based on lumen portions, automatic pre-centering of frames based on EEL portions and lumen morphology, etc.) for example, measured from intravascular imaging or form angiography data), automated measurement procedures (e.g., automated plaque burden measurements), display procedures (e.g., thresholded displays, color-encoded displays, registered overlays, etc.), user inputs procedures (e.g., user selectable thresholds), and any other novel and beneficial procedures in and of themselves disclosed herein, can be applied to any imaging modality, including intravascular ultrasound, Coronary computed tomography angiography (CCTA), magnetic resonance imaging MRI), etc. For example, in some embodiments of the present disclosure, automatic detection and measurement of OCT derived EEL diameters and/or plaque burden estimates can be co-registered to an angiography image, and overlayed on a UI display port (e.g., an angiography UI display port). For example, in some embodiments of the current invention, automatic detection and measurement of IVUS derived EEL diameters and/or plaque burden estimates can be co-registered to an angiography image, and overlayed on a UI display port (e.g., an angiography UI display port). For example, in some exemplary embodiments of the present disclosure, automatic detection and measurement of multimodal, OCT-NIRS derived, EEL diameters and/or plaque burden estimates can be co-registered to angiography-derived (e.g., CCTA-derived) lumen diameters or vascular characteristics (e.g., CCTA-derived calcium features), and overlayed on a UI display port (e.g., and a longitudinal representation of the intravascular and/or extravascular data).

In yet further exemplary embodiments of the present disclosure, automatically detected (e.g., measured) EEL shape (e.g., angle) and size (e.g., diameter) can inform and improve an image-derived physiology measurement (e.g., a fractional flow reserve (FFR) measurement, a virtual fractional flow reserve (vFFR) measurement). For example, an angiography-derived FFR measurement can be improved by incorporating information about the EEL diameter and/or plaque burden. For example, an OCT-derived FFR measurement can be improved by incorporating information about the EEL diameter and/or plaque burden. In some exemplary embodiments of the present disclosure, angiography-derived coronary assessments (ADCA) can be improved using an intravascular detected EEL measurement (e.g., Angiography-derived: structural measurements (e.g., stenosis measurement), absolute or relative coronary flow (CF), Index of microcirculatory resistance (IMR), coronary flow reserve (CFR), coronary microvascular resistance (CMR), etc.). For example, an automatically detected EEL measurement from intravascular OCT and/or IVUS data can be used to re-calibrate an automatically detected lumen measurement from extravascular data (e.g., an x-ray angiography computed lumen measurement), based on, for example the difference or ratio of an automatically detected lumen to automatically detected EEL, for example from intravascular data.

### Technical Description of Exemplary Optimized Plaque Burden Measurements

According to additional exemplary embodiments of the present disclosure, an exemplary optimized plaque burden measurement can be facilitated using an exemplary procedure for determining EEL location when the boundary is obfuscated by lipid, calcium, and other high optically scattering medium. For example, EEL boundary can be enhanced by an OCT frame to frame co-registration that uses visible EEL from neighboring frames to infer its location behind plaque (e.g., based on multiple pre-processing steps). The exemplary OCT co-registration can be, e.g., accomplished by exemplary procedures for correcting for catheter de-centering, precession, vessel tortuosity and NURD. In some exemplary embodiments of the present disclosure, OCT co-registration can be aided by angiography detected lumen morphology characteristics (e.g., tortuosity) and angiography detected intravascular catheter positions (e.g., radiopaque marker positions, imaging positions, imaging path, etc.). In some exemplary embodiments according to the present disclosure, the OCT images can be decentered and/or centered through constrained least-squares elliptical fitting of the artery boundary in every frame based on either OCT data alone, or the OCT data in reference to the angiography derived information (e.g., the imaging path).

In some exemplary embodiments of the present disclosure, angular corrections for NURD and precession can be accomplished using an exemplary speckle analysis and polar phase de-correlation, respectively. For example, the EEL boundary from neighboring frames can be fused with the detected EEL boundary in a given frame using an interpolation (e.g., an optimization-based interpolation, a machine learned interpolations, etc.) for a smooth, low eccentricity curve. Further, e.g., EEL boundary estimation can be further enhanced through a context-based inference using automatically detected lipid, calcium, and side-branches.

According to various exemplary embodiment of the present disclosure, an exemplary EEL detection can be trained/informed based on co-registered IVUS and histology images which shows the EEL boundary behavior behind plaques that would obscure the EEL in optical imaging. In some exemplary embodiments of the present disclosure, side-branches (e.g., side-branches which exceed a certain diameter) are used to adjust the EEL boundary (e.g., in a stepwise fashion, based on a priori information, etc.). For example, such automatically detected information can be used to constrain which frames are used to infer EEL position from neighboring frames, as can be understood that vessel size decreases after significant side branches (e.g., based on Murray's law). The exemplary procedure can infer EEL in frames with small side-branches which would give inaccurate measurements using a single-frame analysis.

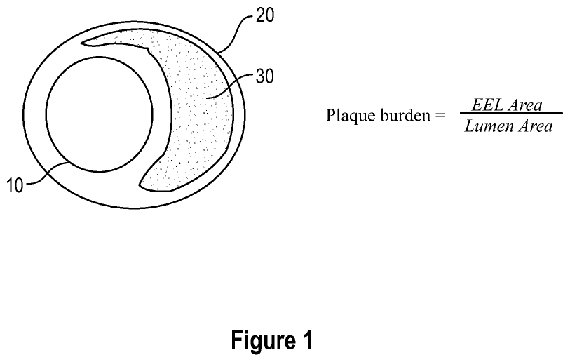

FIG. 1 shows an illustration to assist with an explanation of a burden of plaque 10 shown therein (i.e., a plaque burden). As illustrated in FIG. 1, the ratio of an area of a lumen 30 to EEL 20 area can describe the plaque burden (e.g., at a specific longitudinal cross section along a coronary vessel). In some exemplary embodiments of the present disclosure, the plaque burden can be determined or calculated as any ratio or relation between lumen area and/or plaque area and EEL area. In some exemplary embodiments of the present disclosure, a plaque burden measurement can be a volumetric measurement using relations in volumes (3D) instead of the relations of areas (2D). In some exemplary embodiments of the present disclosure, a specific plaque risk and/or patient risk can be provided based on a volumetric assessment of the plaque along the length of the diseased portion (e.g., the total volume of the plaque).

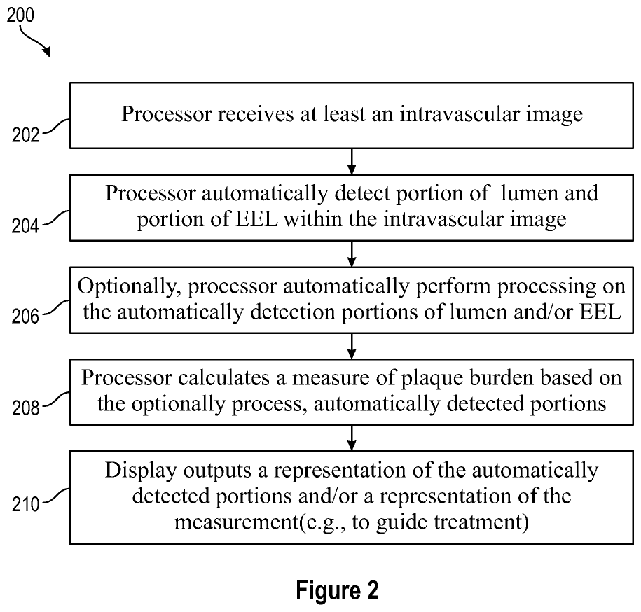

FIG. 2 illustrates a flow diagram workflow or diagram 200 which can be implemented by an exemplary system configured to automatically calculate a plaque burden measurement based on automatically detected portions of lumen and EEL, within an intravascular image, and further display some representation of the automatically detected portions and/or the measurement, in accordance with exemplary embodiments of the present disclosure. For example, as shown in FIG. 2, in procedure 202, at least one processor (e.g., a system containing such processor(s)) can receive synchronized extravascular images and intravascular images of coronary vasculature (e.g., IVOCT images). In procedure 204, the processor automatically detects portion of Lumen and portion of EEL. In procedure 206 the processor(s) can, optionally, automatically perform processing on the automatically detected portions of lumen and/or EEL (e.g. interpolation across frames, e.g., constrained interpolation, e.g., registration), for example to improve the performance. (e.g., accuracy) of the automatic detection (e.g., improve automatic calculation of EEL area). In procedure 208, the processor(s) can calculate or otherwise determine a measure of plaque burden based on the optionally processed, automatically detected portions. In procedure 210, the processor(s) can control a display to output or otherwise display a representation of the automatically detected portions and/or a representation of the measurement (e.g., to guide treatment).

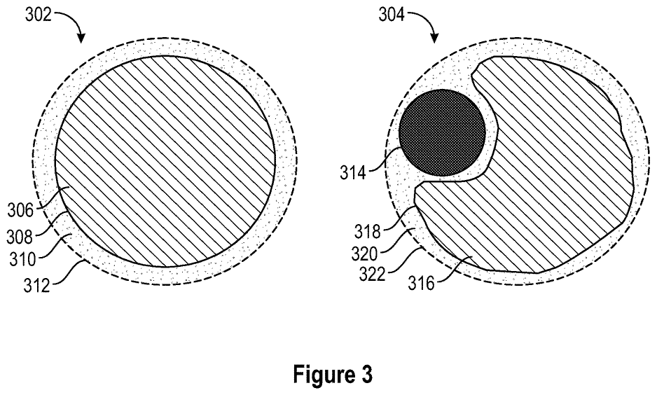

FIG. 3 shows a set of exemplary illustrations of automatically detected portions of an intravascular image, associated with the plaque burden, with and without the presence of plaque (e.g., an atherosclerotic plaque with high plaque burden and high risk). For example, a first vessel cross section 302 and a second vessel cross section 304 with automatically detected features (e.g., portions of lumen, e.g., portions of EEL) are shown for comparison purposes. In the first vessel cross section 302, an automatically measured lumen area 306 defined by an automatically detected lumen perimeter 308 can be contained within a relatively healthy vessel wall 310. The automatically measured lumen area 306 can further contained within an automatically detected EEL perimeters 312. The automatically detected perimeters can be used to automatically measure area for the various vascular regions (e.g., EEL perimeter can be used to automatically measure EEL area). A vessel with such a cross section as shown in item 302 can have a low plaque burden and, therefore, a low corresponding risk. In the second vessel cross section 304, a large plaque 314 can be automatically detected and/or measured within the lumen wall 320. An automatically measured plaque burden can be calculated based on any combination of the automatically measured or detected plaque 314 (e.g., plaque perimeter, plaque area, etc.), the automatically measured lumen area 316, the automatically detected lumen perimeter 318 and/or the automatically detected EEL perimeter 322.

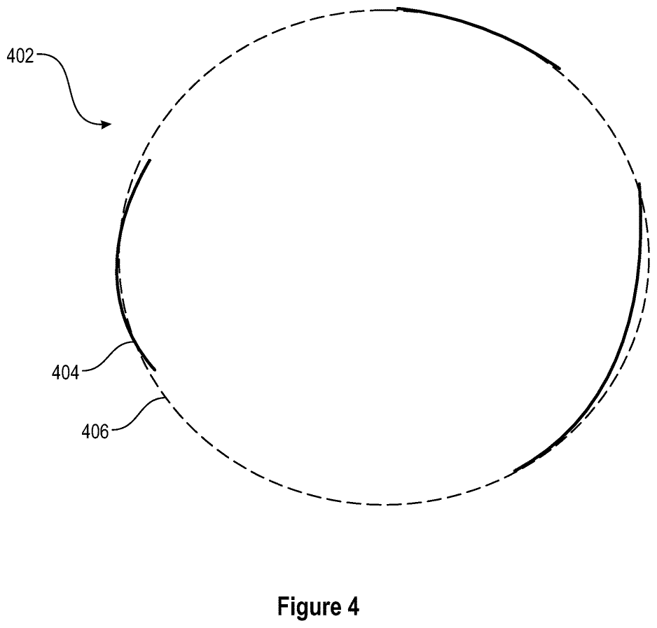

FIG. 4 shows an illustration of an exemplary automatic processing (e.g., interpolation) of automatically measured EEL areas, using automatically detected portions (e.g., contours, e.g., perimeters) of the EEL within an intravascular image according to the exemplary embodiment of the present disclosure. As shown in FIG. 4, an intravascular cross section 402 is illustrated with automatically detected portions of EEL 404 (e.g., visible portions of EEL within an image) and automatically inferred portions of EEL 406 (e.g., not visible portions of EEL within an image). In some exemplary embodiments of the present disclosure, visible portions of the EEL within an intravascular image (e.g., IVOCT image) can be automatically detected (e.g., segmented) using a machine learned detection network (e.g., a convolutional neural network) and automatically interpolated within the transverse direction, (e.g., in the axis of the image, e.g., interpolated in 2D) to further infer the EEL contour (e.g., to improve EEL area measurement accuracy). In some exemplary embodiments of the present disclosure, an interpolation (e.g., smoothing, averaging, etc.) can be performed in the longitudinal direction and/or the transverse direction (e.g., interpolated in 3D) to further infer the EEL contour and/or to provide a volumetric plaque burden calculation (e.g., to improve EEL area measurement accuracy). In some exemplary embodiments of the present disclosure, a plaque burden measurement can be performed only the visible and automatically detected portions of the EEL can be adequate (e.g., above a threshold, detected in sections covering greater than a 30 degrees, detected in sections covering greater than a 60 degrees, detected in sections covering greater than a 90 degrees, detected in sections covering greater than a 180 degrees, detected in sections covering greater than a 220 degrees, etc.). For example, if a small portion of EEL is detected on, for example, opposite sides of the cross section, a threshold can be applied based on an interpolation confidence index (e.g., a constrained interpolation, an eccentricity constrained interpolation, etc.). FIG. 4 shows that 3 portions of EEL are detected, and the interpolation can be performed based on the 3 portions. In some exemplary cases, an interpolation can be performed on only 1 or 2 of the automatically detected portions based on an automatically assessed quality index (e.g., confidence index) for each specific portion.

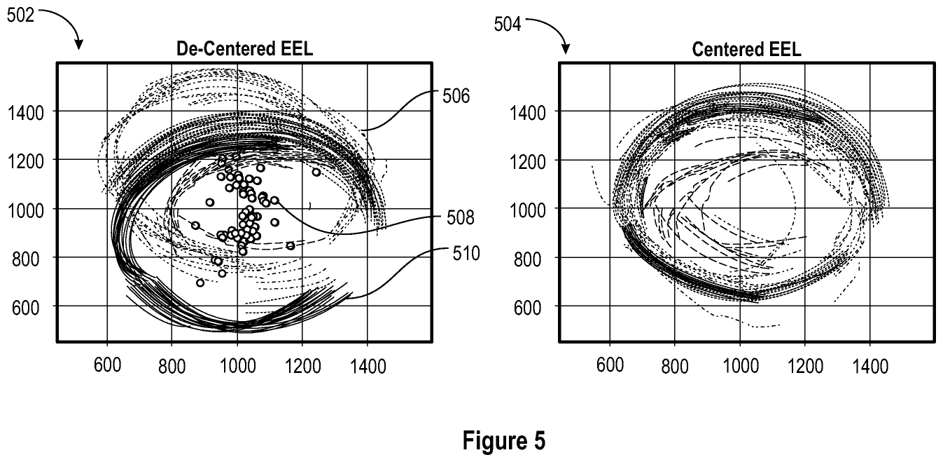

FIG. 5 illustrates exemplary graphs 502, 504 providing exemplary results obtained when centering automatically detected EEL portions based on automatically detected characteristics within an intravascular image, in accordance with the exemplary embodiments of the present disclosure. As shown in FIG. 5, the first graph 502 displays de-centered EEL portions that were automatically detected from an intravascular imaging dataset, according to the exemplary embodiments of the present disclosure. The second graph 504 displays centered EEL portions based on processing and centering methods (e.g., automatic processing and centering methods), according to the exemplary embodiments of the present disclosure. For example, in each intravascular image, automatically detected portions of vascular lumen (not shown in FIG. 5) and/or EEL 506 provide don the first graph 502 can be processed and grouped into sections. Such exemplary sections 510 are depicted by greyscale within each of the graphs 502, 504. Each portion or section can be centered based on an automatically measured vessel centers 508, which can be derived based on automatically detected lumen portions and/or EEL portions and/or plaque portions. In some exemplary embodiments of the present disclosure, lumen and/or EEL inference can comprise an interpolation (e.g., in 2D) before the vessel centering has been applied, for example, to improve centering performance. In some exemplary embodiments of the present disclosure, lumen and/or EEL inference comprises interpolation (e.g., interpolation in 3D) after the vessel centering has been applied, for example, to improve interpolation performance and/or downstream measurement performance (e.g., plaque burden measurement performance).

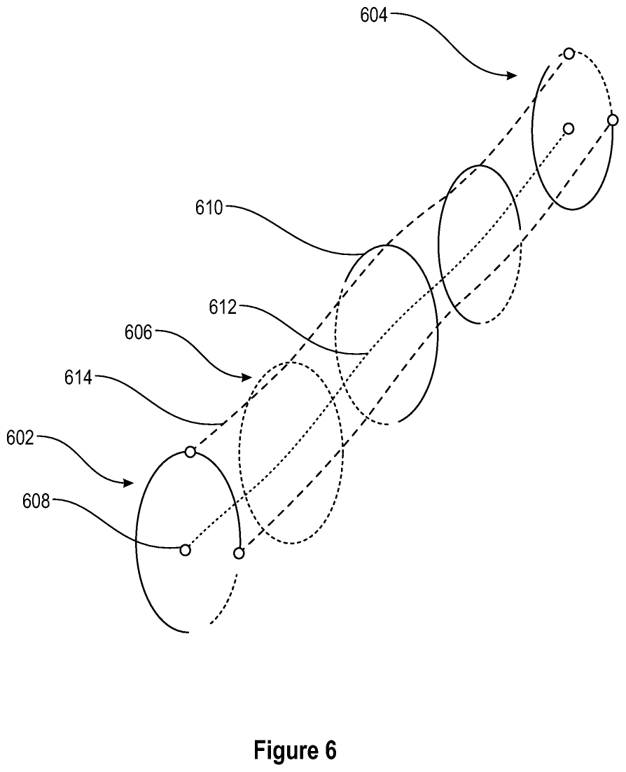

FIG. 6 shows an illustration of ND processing and interpolation of EEL based on automatically detected characteristics within an intravascular image, in accordance with exemplary embodiments of the present disclosure. For example, automatically detected portions of EEL across several images beginning at a first image 602 and ending at a second image 604 can utilize complex processing (e.g., centering) and interpolation to improve automatic EEL inference in other frames such as a frame with poor EEL visibility 606. FIG. 6 illustrates dark full lines which show automatically detected portions of EEL 610 in several intravascular images, dashed lines show automatically inferred portions of EEL 614 and thin dotted lines show automatically inferred a vessel center axis 612 based on image-based automatically inferred vessel centers 608. As shown in FIG. 6, EEL can be visible in one frame at a single circumferential position, although invisible at the same circumferential position in the following frame. For example, EEL can be completely invisible in one frame altogether.

The systems, methods and computer-accessible medium according to the exemplary embodiments of the present disclosure can be used to address such invisible portions by automatic 3D centering (e.g., weighted centering) and interpolation (e.g., machine learned interpolation) that take into account vessel morphology (e.g., plaque locations, e.g., side branch locations, e.g., stent locations) in relation to the position of the imaging probe. In FIG. 6, it is shown that automatically detected portions may be longitudinally offset in the transverse direction and this offset may be caused by a probes position within the vessel or by vessel tortuosity, or a combination of such factors along with many others (e.g., lumen protrusion caused by plaque, e.g., myocardial bridges). As is described herein, automatically processing, and centering automatically detected features in a longitudinal context (e.g., 3D context) can facilitate an improved vascular feature detection (e.g., EEL position detection), and therefore improve accuracy of vascular measurements (e.g., plaque burden measurements).

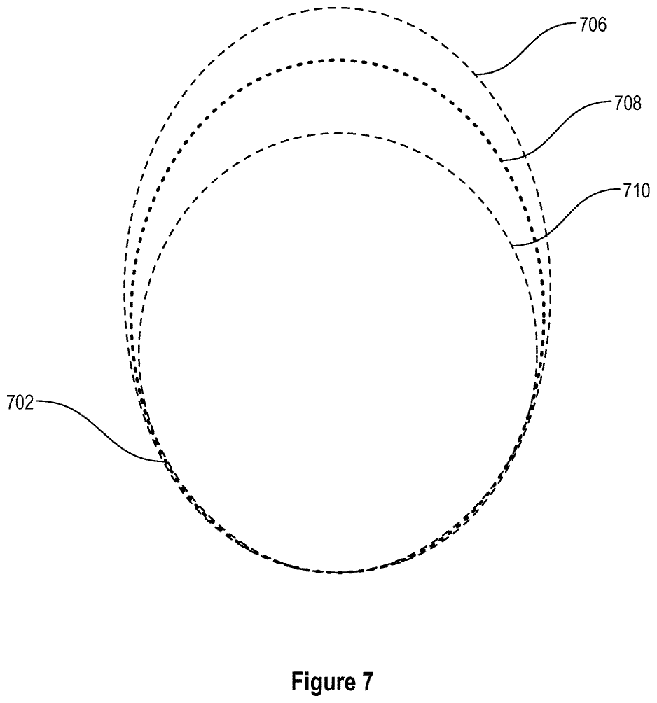

FIG. 7 shows an illustration of multi-dimensional (ND) processing (e.g., 3D, 4D where one dimension may be time, etc.) and interpolation of EEL using an ellipticity constraint that has been optimized and/or machine learned. In an exemplary embodiment of the present disclosure, as shown in FIG. 7, an automatically detected portion of EEL 702 can be interpolated using a high ellipticity constraint 706, a low ellipticity constraint 710 and an optimized interpolation constraint 708. An optimized interpolation constraint can be optimal based on the vessel-specific morphology, patient specific based on patient history, or coronary specific based on other modality imaging (e.g., X-ray angiography, CTA, etc.). In some exemplary embodiments of the present disclosure, an optimized interpolation constraint can be or include a machine learned constraint. In some embodiments of the present disclosure, a machine learned interpolation constraint may be trained using images and/or annotations from a secondary imaging modality (e.g., another intravascular imaging modality, a histology imaging modality, etc.). For example, during training, at least partially registered data (e.g., annotations) from the secondary imaging modality can be used as ground-truth targets for the intravascular imaging modality (e.g., the second imaging modality would not be required for prediction).

According to some embodiments of the present disclosure, an optimized interpolation constraint can be physically-informed constraint (e.g., based on mechanical characteristics of human vasculature). In further exemplary embodiments of the present disclosure, an optimized interpolation constraint can be informed based on adjacent images in the longitudinal direction, or automatically detected vascular objects, such as plaques (e.g., plaques with known hardness). For example, in some exemplary embodiments of the present disclosure, an automatic plaque detection and/or characterization procedure can be used to provide the type of a plaque to adjust the constraint, based on whether a plaque is lipid based or calcium based. In some additional exemplary embodiments of the present disclosure, the shape of an automatically detected lumen can also be used to inform the interpolation constraint. For example, when the lumen wall protrudes (e.g., protrudes eccentrically) into the lumen, a constraint can be adjusted to facilitate a higher ellipticity due to the increased pressure on the EEL in that circumferential direction. In additional exemplary embodiments of the present disclosure, a temporal data (e.g., 2D image data with a temporal dimensionality, 3D image data with a temporal dimensionality, etc.) is used to improve automatic EEL detection and interpolation. For example, multiple longitudinal imaging pullbacks from different timepoints (e.g., successive, pre-PCI and post-PCI, etc.) can be used (e.g., after registration) to improve EEL detection and inference and/or to measure temporal characteristics about the EEL (e.g., biomechanical characteristics) and/or plaque burden.

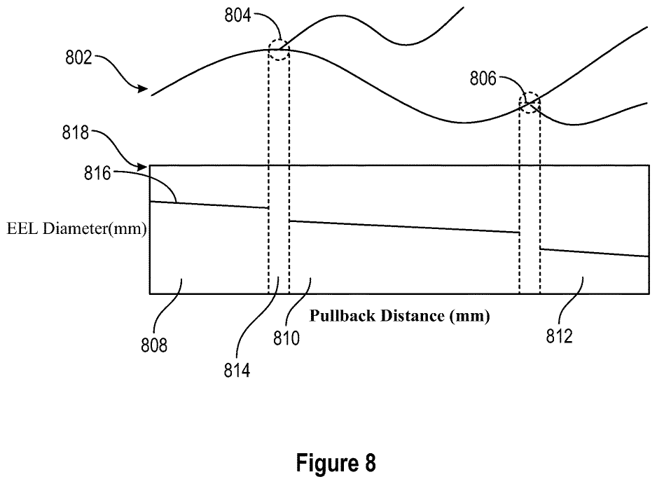

FIG. 8 shows an exemplary graph and illustration of ND processing and interpolation of EEL diameter within continuous vascular regions, defined by automatically detected vascular objects, according to the exemplary embodiment of the present disclosure. As illustrated in FIG. 8, a blood vessel 802 can comprise an automatically detected side-branches vessel 804, 806. The interpolation of an automatically detected and/or inferred EEL and/or measured EEL diameter can only be interpolated between side-branches in sections. The exemplary graph 818 of EEL diameter versus pullback distance (e.g., from an intravascular imaging pullback) EEL diameter decreases along the length of an artery monotonically and can also decrease in a stepwise fashion at the location of major side branches and this effect can make interpolation less accurate, if not accounted for. In some exemplary embodiments of the present invention, a first interpolation section 808 can extend up until the first side-branch 804, a second section 810 can extend from the first side-branch to the second side branch 806, and a third section 812 can extend past the second side branch. Each of these sections 810, 812 can be interpolated over. The exemplary interpolation in this example can refer to interpolating automatically detected portions of vascular structures (e.g., lumen, EEL, etc.), and it can also refer to interpolating over automatic measurements, such as an automatically measured EEL diameter 616 (e.g., where sections are interpolated over disparately). According to some exemplary embodiments of the present disclosure, a designated section can be ignored all together during sectioned (e.g., stepwise) interpolation, such as the location of a detected side-branch 614.

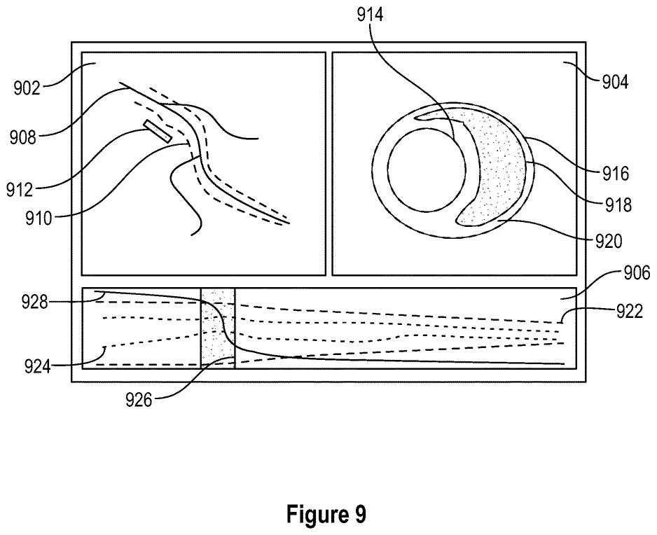

FIG. 9 shows a set of exemplary illustrations of an exemplary multi-panel user interface (UI) for PCI planning and post-PCI assessment, according to exemplary embodiments of the present invention. For example, a multi-panel UI is illustrated for displaying automated outputs during PCI planning and during post-PCI assessment. The exemplary UI can include an angiography display window 902, an intravascular window 904, and a longitudinal representation window 906. The coronary vasculature 908 is provided in the angiography display window 902 and displayed along with a registered representation of longitudinal EEL diameter overlays 910 and a registered threshold representation overlay 912 (e.g., depicting the location of high plaque burden above a threshold). The overlays 910, 912 can be automatically generated from the intravascular imaging data and registered to each other according to exemplary embodiments of the present disclosure.

As shown in FIG. 9, a transverse cross section of a coronary vessel 920 provided the intravascular imaging window 904 can be displayed along with an exemplary representation of the automatically detected and optionally interpolated lumen contour 914, automatically detected and optionally interpolated EEL contour 916, and automatically detected and optionally interpolated plaque contour 918. A longitudinal representation of EEL diameter 922 can be provided within the longitudinal representation window 906, and also including the illustration of a lumen diameter 924 and a pressure gradient (e.g., an image-derived pressure gradient, an angiography derived FFR, etc.) 928. A local thresholded plaque burden representation 926 (also included within the window 906) can be registered along the longitudinal axis with the thresholded plaque burden representation 912 in the angiography window 902. While not shown in FIG. 9, other automatically detected objects from either the angiography data or the intravascular data can be displayed in any of the described UI windows, such as a stent location, a lipid location, a calcium location, a macrophage location, a calcium crystal location, a neointima location, a thin cap location, a weak cap location, a necrotic core location, a fibroatheroma location, a malapposition location. Any of the described objects may also only be displayed if a thresholded measure of their presence is reached.

FIG. 9 shows only exemplary illustration, and it should be understood that a displayed overlay on any of the UI windows can take any shape or form for any automatically detected, processed, or measured structures according to exemplary embodiments of the present disclosure. Any of the described windows can also be occupied by a 3D visualization of a longitudinal structure. EEL overlays along with thresholded plaque burden measurement overlays greater than a pre-defined value.

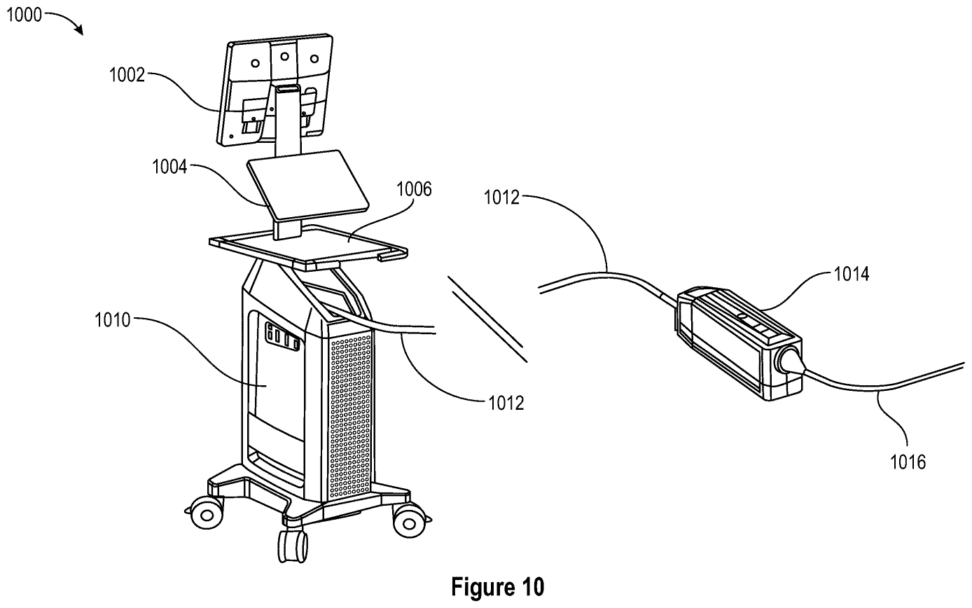

FIG. 10 illustrates a system diagram of an intravascular imaging system 1000, according to an exemplary embodiment of the present disclosure. For example, the exemplary imaging system 1000 can comprise a display configured to acquire intravascular data and register the intravascular data with extravascular data. In some exemplary instances, the intravascular data can comprise one or more intravascular images of a blood vessel. In some exemplary cases, the extravascular data can comprise one or more extravascular images (e.g., x-ray angiogram, MRI, etc.) of blood vessel shape, physiology, anatomy, or any combination thereof. In some exemplary cases, the imaging system can comprise a computer system 1006 to process intravascular, extravascular, user interaction, or any combination thereof data.

The user interaction data can comprise a user inputting data into the imaging system 1000 where the data can comprise patient information, landmark designation, selecting system operation modes, image processing functions, or any combination thereof. In some exemplary cases, a user can input data into the imaging system 1000 with a mouse and/or keyboard electrically coupled with the computer system 1006. A user can visualize a view configured (i.e., user interface) to input data into the system via a first monitor 1002 and/or a second monitor 1004. In some exemplary cases, the first monitor 1002 and/or the second monitor 1004 can comprise a touchscreen interface and keyboard for interacting, acquiring, or any combination thereof actions conducted on the intravascular and/or extravascular data. In some exemplary cases, the user interaction data can comprise data resulting from a user interacting with the extravascular and/or intravascular data (e.g., rotating, zooming in, adjusting contrast, adjusting brightness, measuring a distance, etc.). The computer system 1006 can include or be in communication with an electronic display (e.g., the first monitor 1002 and/or the second monitor 1004) that comprises one or more view configurations (i.e., user interface (UI)), described elsewhere herein, for viewing the intravascular data, extravascular data, a registered and/or combination of the intravascular and extravascular data, or any combination thereof.

In some exemplary cases, the computer system (1006) can comprise an input interface 1005, where the input interface 1005 can comprise one or more input points and/or ports electrically coupled with the computer system 1006. The input interface 1005 can receive one or more data and/or streams of data from one or more imaging systems. For example, the input interface 1005 can receive an x-ray angiography data, where the computer system 1006 can then register the x-ray angiography data with the intravascular data. In some exemplary cases, the input interface 1005 can receive angiography-derived physiology, MRI, computed tomography, spatial positional, intravascular sensor (e.g., intravascular physiology), or any combination thereof data from one or more medical devices to be displayed and/or registered to the intravascular data. In some exemplary instances, the input interface 1005, can receive the data to register to the extravascular data, as described elsewhere herein, wirelessly through an ad-hoc WIFI, Bluetooth, radiofrequency, or any combination thereof wireless communication platform.

In some exemplary embodiments of the present disclosure, the computer system 1006 can process data with one or more processors. In some exemplary instances, the one or more processors can comprise processors of one or more graphical processing units, integrated circuit, or any combination thereof processors. The graphical processing units facilitate processing complex large datasets due to their highly parallel processor architecture. For example, processing data with one or more graphical processing units provides the system with the capability of registering the intravascular data with a real-time stream of extravascular data, otherwise not achieved with traditional multi-core processors.

In some additional exemplary embodiments of the present disclosure, the computer system 1006 can be configured to process the intravascular and extravascular data and/or images. The computer system 1006, can comprise a central processing unit and/or graphical processing (CPU and/or GPU, also “processor” and “computer processor” herein), which can be a single core or multi core processor, or a plurality of processor for parallel processing. The computer system 1006 may further comprise memory or memory locations (e.g., random-access memory, read-only memory, flash memory), electronic storage unit (e.g., hard disk), communications interface (e.g., network adapter) for communicating with one or more other devices, and peripheral devices, such as cache, other memory, data storage and/or electronic display adapters. The memory, storage unit, communications interface, and peripheral devices (e.g., mouse, keyboard, etc.) can be in communication with the CPU and/or GPU through a communication bus (solid lines), such as a motherboard. The storage unit can be a data storage unit (or a data repository) for storing data. The computer system 1006 can be operatively coupled to a computer network (“network”) with the aid of the communication interface 1408. The network can be the Internet, an internet and/or extranet, or an intranet and/or extranet that is in communication with the Internet. The network may, in some exemplary cases, be a telecommunication and/or data network. The network can include one or more computer servers, which can facilitate distributed computing, such as cloud computing. The network, in some exemplary cases with the aid of the computer system 1006 can implement a peer-to-peer network, which can facilitate devices coupled to the computer system 1006 to behave as a client or a server.

The CPU and/or GPU can execute a sequence of machine-readable instructions, which can be embodied in a program or software. The instructions can be directed to the CPU and/or GPU, which may subsequently program or otherwise configured the CPU/GPU to acquire data and/or process data produced by the imaging system described elsewhere herein.

In some exemplary embodiments according to the present disclosure, the computer system (1006) central processing unit and/or graphical processing unit may execute machine executable or machine-readable code that can be provided in the form of software to transfer data generated by the imaging system to a network and/or cloud for further processing, classification, data clustering, or any combination thereof operations. In some exemplary instances, the data can comprise the intravascular and/or extravascular data, described elsewhere herein. In some exemplary cases, the data can comprise image pixel data. In some exemplary instances, the pixel data can comprise optical coherence tomography, x-ray angiography, computed tomography, intravascular ultrasound, spectroscopy, MRI, or any combination thereof image pixel data.

In some further exemplary embodiments of the present disclosure, the CPU and/or GPU can be part of a circuit, such as an integrated circuit. One or more other components of the exemplary system can be included in the circuit. In yet further exemplary embodiments of the present disclosure, the circuit can comprise an application specific integrated circuit (ASIC).

The storage unit may store files, such as drivers, libraries, and saved programs. The storage unit may store acquired x-ray angiography, optical coherence tomography, intravascular ultrasound, near infrared spectroscopy, photoacoustic or any combination thereof data and/or images. In some exemplary cases, the intravascular and/or extravascular data and/or images can be stored in the cloud, a medical system electronic medical records (e.g., EPIC), or any combination thereof locations. The computer system 1006, in some exemplary cases can comprise one or more additional data storage units that are external to the computer system 1006, such as located on a remote server that is in communication with the computer system 1006 through an intranet or the internet.

In some exemplary cases, the imaging system 1000 is in electrical and/or optical communication to an imaging probe actuator 1010, and an imaging probe 1012, as seen in the imaging system 1000 can be in electrical and/or optical communication with the imaging probe actuator 1010 through one or more electrical and/or optical communication wires 1008. In some exemplary cases, the imaging probe 1012 can be releasably coupled to the imaging probe actuator 1010, such that a first imaging probe can be removed from the imaging probe actuator and replaced with a second imaging probe.

According to still further exemplary embodiments of the present disclosure, the imaging probe can comprise an intravascular imaging probe. The intravascular imaging probe can comprise an optical coherence tomography, intravascular ultrasound, reflectance, photoacoustic, near infrared spectroscopy, fluorescence, or any combination thereof imaging probes. In some exemplary instances, the imaging probe can obtain, collect, and/or detect intravascular data from an inner lumen and/or body of a blood vessel. In some exemplary cases, the intravascular data can comprise two-dimensional (e.g., circular cross-sectional data), and/or volumetric intravascular data (i.e., one or more two-dimensional circular cross-sectioned data as a function of the length of the optical axis of the imaging probe). In some exemplary cases, the imaging probe can comprise one or more radio-opaque markers and/or indicia that can be visualized on extravascular imaging modalities e.g., x-ray angiography, computed tomography, MRI, or any combination thereof extravascular imaging modalities.

In yet additional exemplary embodiments of the present disclosure, the imaging probe actuator 1010 can rotate and/or translate the imaging probe 1012, to obtain two and/or three-dimensional intravascular datasets. In some exemplary cases, the probe can be rotated by a stepper motor, dc-brushless motor, or any combination thereof motors coupled to an optic rotary joint. In some exemplary embodiments, the imaging probe actuator 1010 can translate the imaging probe 1012 with a stage, where the stage can comprise a linear and/or a planar translational stage. The stage translation and the rotation of the imaging probe actuator 1010 can be set and/or adjusted by a user via the one or more interfaces of the imaging system 1000, described elsewhere herein. In some exemplary embodiments, the stage translation and the rotation of the imaging probe actuator 1010 can be determine and/or set by the system based on pre-set standard values for a particular type of imaging procedure or frequently used settings.

Exemplary embodiments of the systems, computer-accessible medium and methods provided herein, such as the computer system 1006, can be embodied in programming. Various exemplary aspects of the technology can be thought of a “product” or “articles of manufacture” typically in the form of a machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of a computer, processor the like, or associated modules thereof, such as various semiconductor memories, tape drives, disk drives and the like, which can provide non-transitory storage at any time for the software program. All or portions of the software may at times be communicated through the Internet or various other telecommunication networks. Such communications, for example, can facilitate loading of the software from one computer or processor into another, for example, from a management server or host computer into the computer platform of an application server. Thus, another type of media that can bear the software elements includes optical, electrical, and electromagnetic waves, such as used across physical interfaces between local devices, through wired and optical landline networks and over various air-links. The physical elements that carry such waves, such as wired or wireless links, optical links, or the like, also can be considered as media bearing the software. As used herein, unless restricted to non-transitory, tangible “storage’ media, term such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media can include, for example, optical or magnetic disks, such as any of the storage devices in any computer(s) or the like, such as can be used to implement the databases, etc. Volatile storage media can include dynamic memory, such as main memory of such a computer platform. Tangible transmission media includes coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefor can include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instruction to a processor for execution.

In some further exemplary embodiments of the present disclosure, the exemplary system 1100 can comprise a computer system (1006) suitable for implementing machine learning procedures and/or predictive models configured to analyze, process, segment and/or label extravascular and/or intravascular data collected by the imaging system 1000, imaging probe 1012, and imaging probe actuator 1010 described elsewhere herein. In some exemplary cases, one or more intravascular and/or extravascular images can be generated from the intravascular and/or extravascular data. In additional exemplary embodiments of the present disclosure, predictive models e.g., machine learning models and/or machine learning procedures may analyze, extract, condense, reduce, predict, process, classify, segment or any combination thereof operations conducted on the intravascular and/or extravascular data. In some exemplary embodiments according to the present disclosure, the systems disclosed herein may implement one or more machine learning procedures and/or model(s) to identify, classify, process and/or segment regions of interest of intravascular and/or extravascular data.

In some exemplary instances, the one or more categories and/or features of the data can be provided to one or more treatment parameter machine learning model and/or procedures to determine suggested treatment and/or treatment parameters (e.g., what type of stent to place and where spatially to best place the stent to achieve clinical efficacy of treatment). The one or more treatment parameter machine learning models can be trained with prior features and corresponding treatment efficacy (i.e., whether any complications ensued after clinical intervention with the system) to generate one or more trained treatment parameter machine learning models to predict efficacious treatments. The spatial orientation of labeled features and their relationship to one another can be other features determined and considered by the treatment parameter machine learning models. In some exemplary cases, the one or more categories and/or features of data for extravascular data can comprise background data, healthy blood vessel morphology, stenotic blood vessel morphology, or occluded blood vessel. In some exemplary cases, the one or more categories of data for the intravascular data can comprise blood vessel tissue of the epithelium, blood vessel tissue of the intima, blood vessel tissue of the adventitia, plaque within the blood vessel tissue, EEL, plaque burden, vulnerable plaque within the blood vessel tissue, or any combination thereof. In some exemplary cases, the one or more categories and/or features of intravascular data can comprise spectroscopic (e.g., in the near infrared) signature of the intravascular blood vessel tissue. For example, the one or more categories and/or features may classify the composition of plaque of the blood vessel based on its spectroscopic signature. In some exemplary cases, the one or more categories can comprise a calcium or a lipid spectroscopic signature. In some exemplary instances, the machine learning model and/or procedure may pre-process the intravascular and/or extravascular data prior to classifying a feature of the data. In some exemplary instances, prep-processing the intravascular and/or extravascular data can comprise de-noising, smoothening, averaging, sharpening, brightness and/or contrast adjustment, or any combination thereof mathematical manipulation of the data. In some exemplary cases, the features and/or categories of the intravascular and/or extravascular data can be extracted without a pre-processing step/procedure.

In some exemplary cases, machine learning procedure procedures can extract and/or draw relationships between features as conventional statistical techniques may not be sufficient. In some exemplary cases, machine learning procedures can be used in conjunction with conventional statistical techniques. In some exemplary cases, conventional statistical techniques can provide the machine learning procedure with pre-processed features.

In some exemplary embodiments according to the present disclosure, any number of features can be classified by the machine learning procedure. The machine learning procedure may classify at least 1 feature. In some exemplary cases, the plurality of features can include between about 1 feature to 5 features. In some exemplary cases, the plurality of features can include between about 5 features to 10 features. In some exemplary cases, the plurality of features can include between about 10 features to 50 features.

In some exemplary embodiments according to the present disclosure, the machine learning procedure can be, for example, an unsupervised learning procedure, supervised learning procedure, or a combination thereof. The unsupervised learning procedure can be, for example, clustering, hierarchical clustering, k-means, mixture models, DBSCAN, OPTICS procedure, VoxelMorph procedure, anomaly detection, local outlier factor, neural networks, autoencoders, deep belief nets, Hebbian learning, generative adversarial networks, self-organizing map, expectation-maximization procedure (EM), method of moments, blind signal separation techniques, principal component analysis, independent component analysis, non-negative matrix factorization, singular value decomposition, or a combination thereof. The supervised learning procedure can be, for example, support vector machines, linear regression, logistic regression, linear discriminant analysis, decision trees, k-nearest neighbor procedure, neural networks, similarity learning, or a combination thereof. In some exemplary embodiments according to the present disclosure, the machine learning procedure can comprise a deep neural network (DNN). The deep neural network can comprise a convolutional neural network (CNN). The CNN can be, for example, U-Net, ImageNet, LeNet-5, AlexNet, ZFNet, GoogleNet, VGGNet, ResNet18 or ResNet, etc. Other neural networks can be, for example, deep feed forward neural network, recurrent neural network, LSTM (Long Short-Term Memory), GRU (Gated Recurrent Unit), Auto Encoder, variational autoencoder, adversarial autoencoder, denoising auto encoder, sparse auto encoder, Boltzmann machine, RBM (Restricted BM), deep belief network, generative adversarial network (GAN), deep residual network, capsule network, or attention/transformer networks, etc.

In some exemplary instances, the machine learning model can comprise clustering, scalar vector machines, kernel SVM, linear discriminant analysis, Quadratic discriminant analysis, neighborhood component analysis, manifold learning, convolutional neural networks, reinforcement learning, random forest, Naive Bayes, gaussian mixtures, Hidden Markov model, Monte Carlo, restrict Boltzmann machine, linear regression, or any combination thereof.

In some exemplary cases, the machine learning procedure can include ensemble learning procedures such as bagging, boosting and stacking. The machine learning procedure can be individually applied to the plurality of features extracted.

In some exemplary embodiments according to the present disclosure, the exemplary systems and/or methods can apply one or more machine learning procedures and/or an ensemble of machine learning procedures.

In some exemplary embodiments according to the present disclosure, the machine learning procedure can include and/or utilize a variety of parameters. The variety of parameters can be, for example, learning rate, minibatch size, number of epochs to train for, momentum, learning weight decay, or neural network layers etc.

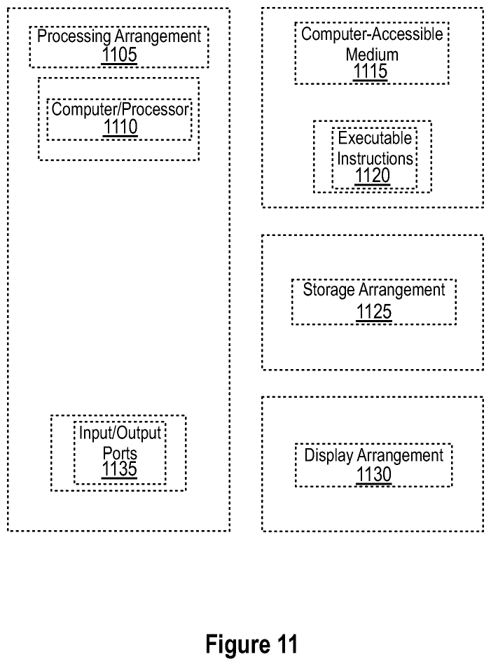

FIG. 11 shows a block diagram of another exemplary embodiment of a system according to the present disclosure. For example, exemplary procedures in accordance with the present disclosure described herein can be performed by a processing arrangement and/or a computing arrangement (e.g., computer hardware arrangement) 705. Such processing/computing arrangement 1105 can be, for example entirely or a part of, or include, but not limited to, a computer/processor 1110 that can include, for example one or more microprocessors, and use instructions stored on a computer-accessible medium (e.g., RAM, ROM, hard drive, or other storage device).

As shown in FIG. 11, for example a computer-accessible medium 1115 (e.g., as described herein above, a storage device such as a hard disk, floppy disk, memory stick, CD-ROM, RAM, ROM, etc., or a collection thereof) can be provided (e.g., in communication with the processing arrangement 1105). The computer-accessible medium 1115 can contain executable instructions 1120 thereon. In addition, or alternatively, a storage arrangement 1125 can be provided separately from the computer-accessible medium 1115, which can provide the instructions to the processing arrangement 1105 so as to configure the processing arrangement to execute certain exemplary procedures, processes, and methods, as described herein above, for example.

Further, the exemplary processing arrangement 1105 can be provided with or include an input/output ports 1135, which can include, for example a wired network, a wireless network, the internet, an intranet, a data collection probe, a sensor, etc. As shown in FIG. 11, the exemplary processing arrangement 1105 can be in communication with an exemplary display arrangement 1130, which, according to certain exemplary embodiments of the present disclosure, can be a touch-screen configured for inputting information to the processing arrangement in addition to outputting information from the processing arrangement, for example. Further, the exemplary display arrangement 1130 and/or a storage arrangement 1125 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

What is claimed is:

1. An apparatus, comprising:
an imaging catheter configured to acquire one or more intravascular images; and
at least one computer processing configuration configured to:
a) automatically detect one or more first portions of an external elastic laminae,
b) automatically infer or determine one or more second portions of an external elastic laminae, and
c) automatically determine at least one measurement of a diameter of the external elastic laminae diameter based on the first portions and the one or more second portions.

2. The apparatus of claim 1, wherein the least one computer processing configuration is configured to automatically estimate a plaque burden from at least one of the one or more first portions or the one or more second portions.

3. The apparatus of claim 2, further comprising a display device which is configured to display a representation of the external elastic laminae, or the plaque burden based on a viewport within a user interface.

4. The apparatus of claim 3, wherein the display device displays the representation upon reaching a predetermined threshold of the plaque burden.

5. The apparatus of claim 1, wherein the one or more intravascular images are optical coherence tomography (OCT) images received from an OCT system with a sensitivity that is greater than about 100 dB.

6. The apparatus of claim 5, wherein the one or more second portions are determined or inferred using a machine learning procedure.

7. The apparatus of claim 6, wherein the machine learning procedure determines or infers the one or more invisible portions by an interpolation from neighboring frames.

8. A method for determining or providing one or more measurements of an arterial wall, comprising:
obtaining an image of at least one section of the arterial wall;
automatically detecting one or more visible portions of an arterial external elastic laminae of the at least one section;
automatically determining or estimating one or more invisible portions of the arterial external elastic laminae based on the one or more automatically detected visible portions; and;
automatically combining the visible and invisible portions to provide the one or more measurements of the external elastic laminae.

9. The method of claim 8, wherein the one or more invisible portions are determined or estimated using a machine learning procedure.

10. The method of claim 9, wherein the machine learning procedure determines or estimates the one or more invisible portions by an interpolation from neighboring frames.

11. The method of claim 8, further comprising providing one or more further measurements of a plaque burden.

12. The method of claim 11, further comprising displaying a representation of at least one of the external elastic laminae or the plaque burden on a viewport of a user interface.

13. The method of claim 12, wherein the representation is displayed upon reaching a predetermined threshold of the plaque burden.

14. The method of paragraph 1, wherein, the intravascular imaging is performed using an optical coherence tomography (OCT) system with a sensitivity greater than 100 dB.

15. An apparatus, comprising:
an imaging catheter configured to obtain an image of at least one section of an arterial wall; and
at least one computer processing configuration configured to:
i. automatically detect one or more first portions of an arterial external elastic laminae of the at least one section,
ii. automatically detecting one or more second portions of an arterial lumen, and
iii. determine or estimate a plaque burden by processing the detected one or more first portions and the one or more second portions.

16. The apparatus of claim 15, further comprising a display device configured to display a representation of the plaque burden on a viewport of a user interface.

17. The apparatus of claim 16, wherein the representation is displayed when a predetermined threshold of the plaque burden is reached.

18. A method for determining or providing one or more measurements of an arterial wall, comprising:
obtaining an image of at least one section of the arterial wall;
automatically detecting one or more first portions of an arterial external elastic laminae of the at least one section;
automatically detecting one or more second portions of the arterial wall; and
determining or estimating a plaque burden by processing the detected one or more first portions and the one or more second portions.

19. The method of claim 18, further comprising displaying a representation of the plaque burden on a viewport of a user interface.

20. The method of claim 19, wherein the representation is displayed when a predetermined threshold of the plaque burden is reached.

21. A method for determining or providing one or more measurements of a coronary artery, comprising:
obtaining an image of at least one portion of the coronary artery; and
with a machine learned procedure, directly determining or estimating at least one measure of a plaque burden based on one or more features or information for the at least one portion of the image.

22. An apparatus, comprising:
an imaging catheter configured to obtain an image of at least one section of a coronary artery; and
at least one computer processing configuration configured to directly determining or estimating at least one measure of a plaque burden based on one or more features or information for the at least one portion of the image using a machine learned procedure.

23. A method for determining or providing one or more measurements of a coronary artery, comprising:
obtaining an image of at least one portion of the coronary artery; and
with a machine learned procedure, determining or estimating ae position the external elastic laminae within the at least one portion of the image.

24. The method of claim 23, wherein the image is generated using a first imaging modality, and further comprising training the machine learned procedure is to determine or estimate the position of the external elastic laminae using registered data from a second image modality which is different from the first image modality.

25. The method of claim 24, wherein the registered data has a resolution lower than that of the image from the first imaging modality.

26. The method of claim 23, wherein the registered data was a resolution lower than that of the first imaging modality.

27. The method of claim 23, wherein the first resolution is histology or IVUS.

28. The method of claim 23, wherein the machine learned procedure determines or estimates the position of the external elastic laminae in areas where the external elastic laminae is invisible within the image.

29. The method of claim 20, wherein the machine learned procedure generates further images which are adjacent to the image.

30. An apparatus, comprising:
an imaging catheter configured to obtain an image of at least one section of a coronary artery; and
at least one computer processing configuration configured to directly determining or estimating ae position the external elastic laminae within the at least one portion of the image using a machine learned procedure.

31. The apparatus of claim 30, wherein the image is generated using a first imaging modality, and wherein the machine learned procedure is trained to determine or estimate the position of the external elastic laminae using registered data from a second image modality which is different from the first image modality.

32. The apparatus of claim 31, wherein the registered data has a resolution lower than that of the image from the first imaging modality.

33. The apparatus of claim 30, wherein the registered data was a resolution lower than that of the first imaging modality.

34. The apparatus of claim 30, wherein the first resolution is histology or IVUS.

35. The apparatus of claim 30, wherein the machine learned procedure determines or estimates the position of the external elastic laminae in areas where the external elastic laminae is invisible within the image.
